(19) United States
(12) Patent Application Publication
(10) Pub. No.: US 2022/0001012 A1
(43) Pub. Date: Jan. 6, 2022

(54) NOVEL COMPOUNDS AND USES OF SAME FOR NEAR-INFRARED CHERENKOV LUMINESCENCE IMAGING AND/OR FOR DEEP TISSUE TREATMENT BY CHERENKOV DYNAMIC PHOTOTHERAPY

(71) Applicant: UNIVERSITE DE BOURGOGNE, DIJON (FR)

(72) Inventors: Richard DECREAU, DIJON (FR); Vivian LIORET, NUITS SAINT GEORGES (FR); Yann BERNHARD, SAULXURES-LES-NANCY (FR); Bertrand COLLIN, DIJON (FR)

(73) Assignee: UNIVERSITE DE BOURGOGNE, DIJON (FR)

(21) Appl. No.: 17/288,679

(22) PCT Filed: Oct. 25, 2019
(86) PCT No.: PCT/FR2019/052550

§ 371 Date: Apr. 26, 2021

(51) Int. Cl. A61K 41/00; A61K 49/00; A61K 51/04; C07B 59/00

(52) U.S. Cl. CPC A61K 41/0076; A61K 49/0036; A61K 49/0032; C07B 59/004; A61K 51/0446; A61K 51/0497; A61K 51/0485

(57) ABSTRACT

Compounds of the general structure (I), which includes: a radioactive entity, which is a beta-energy emitter that produces Cherenkov radiation, a fluorophore that absorbs electromagnetic radiation of a wavelength λ ranging from 300 nm to 500 nm; a fluorophore which emits electromagnetic radiation of a wavelength λ ranging from 650 nm to 950 nm and/or is a photosensitizer which produces reactive oxygen species ROSs; and a vector entity, which may be present or absent. Also, the use of these compounds for an application for near-infrared Cherenkov luminescence imaging and/or for the treatment of deep biological tissues by Cherenkov dynamic phototherapy.

The subject of the present invention is novel compounds which can in particular be used for near-infrared Cherenkov luminescence imaging and/or for deep biological tissue treatment by Cherenkov photodynamic therapy.

Imaging-guided surgery is a practice which makes it possible to refine the surgical procedure by making it possible to move toward a complete tumor resection and to minimize the excision of healthy tissues. Several preclinical studies have shown that Cherenkov luminescence imaging (denoted CLI in the remainder of the text), which is an optical imaging, can be successfully used to guide the surgical resection of tumors and of lymph nodes, but also of the detection of cancerous lesions using Cherenkov luminescence endoscopy (CLE)⁽¹⁾. Several clinical studies are ongoing on the preoperative and peroperative use of CLI for various cancers, such as prostate cancer, breast cancer, gastrointestinal cancers and also metastatic lymph nodes⁽¹⁾. By making it possible to improve the accuracy of surgical resection, CLI has the potential to become a groundbreaking technology in cancer surgery.

However, CLI-guided surgery has potential challenges inherently linked to the source used for CLI, namely Cherenkov radiation (denoted CR in the remainder of the text), which in particular has the disadvantage of being a relatively low-intensity signal⁽¹⁾.

The compounds of the invention will in particular make it possible to improve the signal in the tissue transparency zone, for an equivalent amount of radio-pharmaceutical doses. Radiopharmaceuticals are substances which, because of their physicochemical and nuclear characteristics, can be used for the diagnosis and treatment of cancer. One class of radiopharmaceuticals contains beta-energy emitters that can emit the CR that can be used for CLI, which is quite a recent technique (2009)⁽¹⁾ and which is stimulating great interest. CR emits in the ultraviolet (UV) range and the blue range, namely in the range of the electromagnetic spectrum having a wavelength (λ) ranging from 300 to 500 nm (nanometers), where biological tissues are the most opaque and therefore not very transparent.

In order to be able to fully exploit CLI, it is therefore necessary to transfer a part of the CR to the zone of the electromagnetic spectrum where tissues are much more transparent, namely in the near-infrared (IR) zone having a wavelength (λ) ranging from 650 to 900 nm.

The transfer of a part of the CR in the near-IR range has already been described in the literature.

Some authors^{(2,3,4)} have also proposed the use of nanoparticles of the “Quantum Dots” (“QDs”) type which represent effective platforms for performing such a transfer, but which, however, have the drawback of being toxic owing to the presence of cadmium for some of them.

The inventors have for their part proposed the activation of fluorophores by CR in order to transfer a part of this CR into the near-IR range. A fluorophore is a chemical substance capable of emitting fluorescent light after excitation.

More particularly, in the document Bernhard et al. from 2014⁽⁵⁾, “radiochelate-fluorophore” conjugates are described in which the radiochelate is bonded to the fluorophore by a bond. The radiochelate is a beta-energy emitter which produces CR and which allows a transfer of energy of CRET (“Cherenkov Radiation Energy Transfer”) type to the fluorophore which, after having received the CR, emits a fluorescence radiation. However, these radiochelate-fluorophore conjugates only allow modest Stokes shifts, of about approximately 20 nm; consequently, transfer to the near-IR region is not achieved. The Stokes shift represents the difference, in wavelength, between the position of the absorption spectrum peak and that of the emission spectrum peak.

In the document Bernhard et al. from 2017⁽⁶⁾, the inventors have described multimolecular systems comprising several fluorophores. More particularly, these systems comprise a radiometal and two or three fluorophores (fluorophore-1, fluorophore-2 and optionally fluorophore-3) which are not bonded to one another by a bond. The radiometal is a beta-energy emitter which produces CR and which allows CRET-type energy transfer to the fluorophore-1, which in turn will transfer the energy received to the fluorophore-2 by FRET (“Förster Resonance Energy Transfer”) type energy transfer, then the fluorophore-2, after having received the energy from the fluorophore-1, subsequently emits a fluorescence radiation. In the case where three fluorophores are present, the fluorophore-2 transmits the energy received to the fluorophore-3 by FRET-type energy transfer, then the fluorophore-3 subsequently emits a fluorescence radiation.

The drawback of this multimolecular system is in particular a/ that the energy transfer takes place with large losses and that the fluorescence emission is weak (there is therefore a poor energy transfer yield and a low brightness), b/ that it is not unimolecular and therefore it is not possible to imagine an identical biodistribution from one component to the other of such a multimolecular system, and c/ that it is not biovectorized, and therefore not specific for a biological target.

In the document of Bizet et al. from 2018⁽⁷⁾, “fluorophore-1-fluorophore-2” conjugates, activated by an exterior radiation source, are described, the fluorophore-1 absorbing in the UV zone of the spectrum and the fluorophore-2 emitting in the near-IR zone, for an application in cell imaging, and more particularly for visualizing B16F10 melanoma cells. However, the conjugates described in this document are not water-soluble and in addition are not very soluble in organic solvents.

Conjugates of fluorophores of dyad, triad, etc., type, which meet the criterion of excitation in the low wavelengths and of re-emission in the high wavelengths, have been described in the literature^{(10, 11, 12, 13)}. However, these systems are not suitable for CLI and have neither a conjugation site nor a site for introducing a radioactive entity.

A first objective of the invention is to obtain new compounds which can advantageously be used for near-IR CLI.

The term “near-IR CLI” is intended to mean the transfer of CR to the near-IR range.

The properties which are sought by the inventors for such compounds are in particular those of having a high brightness, significant Stokes shifts, namely of approximately 300 to 400 nm, with emissions in the region of 800 nm, and/or good energy transfer yields, namely yields greater than 40%, preferably greater than 50%.

The term “Brightness” is the product of the fluorescence quantum yield “ϕF” and of the molar extinction coefficient “ε” (or probability of absorption): Brightness=#F×ε.

Thus, a brightness considered to be “correct” or “good” can be the result of a good fluorescence quantum yield although the molar extinction coefficient is less good, or vice versa.

Ideally, when the fluorescence quantum yield and the molar extinction coefficient are both good, then the brightness is high.

To satisfy the objective of the invention described above, it would ideally be necessary for all these parameters to be as high as possible for the compounds of the invention in order to “transport” a maximum of Cherenkov photons with the highest efficiency from the UV range to the near-IR range, so that the radiance in the near-IR zone resulting from the CR alone is increased once the compound of the invention is placed in the presence of the CR.

However, if one of these parameters (for example the brightness) drops but the other increases (for example significant Stokes shifts), then the compounds of the invention remain advantageous for their envisioned application.

The term “high” brightness is intended to mean a brightness greater than 10 000 M⁻¹cm⁻¹, and preferably greater than 100 000 M⁻¹cm⁻¹.

A “good” brightness is a brightness ranging from 1000 M⁻¹ cm⁻¹ to 10 000 M⁻¹ cm⁻¹.

Another property sought by the inventors for the compounds of the invention is their good solubility in water and/or solvents, in particular organic solvents.

After intense research studies carried out by the inventors, the latter have developed compounds which meet the needs described above, these compounds comprising one or more radioactive entities producing CR, several fluorophores and optionally a vector molecule, all bonded to one another so as to form a single-molecule structure.

A second objective of the invention is to obtain new compounds which can advantageously be used for deep biological tissue treatment by Cherenkov photodynamic therapy.

Photodynamic therapy, hereinafter denoted by PDT, is a technique used clinically for the adjuvant treatment of cancers in superficial zones which can be accessed by a light source (skin, melanoma, esophagus, head and neck, bladder, prostate), but also for the treatment of acne or else in ophthalmology⁽²⁾.

However, because of the limit of light penetration into the tissues⁽²⁾, conventional PDT makes it possible to reach only the “non-deep” or superficial tissues, which are defined as the tissues where the penetration of light originating from an exogenous source is possible, that is to say a few millimeters to 10 mm at most. PDT does not therefore make it possible to reach deep tissue zones (beyond 10 mm). PDT, in the same way as optical imaging, therefore suffers from the fact that light is absorbed by the tissues and can be used only for surface treatments.

PDT involves more particularly the concomitant use of a photosensitizer and of light at a certain wavelength. A photosensitizer is a compound which, under irradiation, has the capacity to transfer its electronic excitation energy to another compound. In PDT, the role of the photosensitizer is to absorb light and to transfer, to the oxygen present in the organism, the energy thus captured in order to convert it into reactive oxygen species, hereinafter denoted ROSs. The ROSs will react with the tumor cells and destroy them. The photosensitizer, previously injected or applied topically, is intended to concentrate as selectively as possible in the tissues to be treated. The wavelength of the excitation light is adjusted to the absorption spectrum of the photosensitizer. The irradiation then triggers a cascade of chemical reactions which will produce the ROSs. PDT, just like CLI, is therefore an optical technique since it is based on the activation of photosensitizers (anticancer agents) by light.

However, many photosensitizers absorb in the UV/blue region of the electromagnetic spectrum, where biological tissues are not transparent. Other research studies have been carried out on photosensitizers which absorb in the near-IR region of the spectrum where the transparency of the tissues is more pronounced.

The use of CR in order to serve as a light source for PDT has already been described in the literature. The authors Anyanee KamKaew et al.⁽⁹⁾ have thus proposed chlorins (photosensitizers) immobilized on silica nanoparticles which are radiolabeled. These immobilized chlorins, which intrinsically capture the CR, are not however optimized for absorbing CR optimally, and they therefore do not generate a large amount of ROSs. Furthermore, they are not covalently conjugated to a biomolecule, and therefore there is no possibility of selectively reaching one biological target other than another. Finally, the molecular structures proposed by these authors are nanoparticle systems and not molecular systems.

After intense research studies carried out by the inventors, the latter have imagined compounds in which the light source is an integral part of the compounds of the invention. The compounds of the invention comprise a radioactive entity which is conjugated to a photosensitizer. The radioactive entity which emits light (CR) can thus be designed as an onboard light source, since it is entrained with the photosensitizer to the tumor cells. There is therefore no limit to the depth of use of the photosensitizer.

PDT can thus also be used for deep tissues, this is Cherenkov PDT.

The invention will thus advantageously allow the access of PDT to any type of tissue depth, greater than 10 mm and more. The term “deep biological tissue” is intended to mean, according to the invention, tissues which are at a depth ranging from 1 cm to 30 cm under the horny layer of the skin.

In addition, when the photosensitizer thus radiolabeled according to the invention is conjugated to a vector entity targeting cancer tissue receptors, this will make it possible to be selective for cancer tissues on which it is desired to perform deep treatment.

According to a first subject, the invention relates to novel compounds comprising one or more radioactive entities A, one or more fluorophores B, a fluorophore and/or photosensitizer C, and optionally a vector entity D, all bonded to one another so as to form a single-molecule structure.

According to a second subject, the invention relates to the use of these compounds:
for near-IR CLI when C is only a fluorophore,
for the treatment of deep biological tissues by Cherenkov PDT when C is only a photosensitizer,
for near-IR CLI and Cherenkov PDT when C is both a fluorophore and a photosensitizer.

According to a third subject, the invention relates to the method for preparing the compounds of the invention.

A subject of the present invention is more particularly a compound having the following general structure (I):

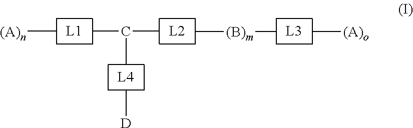

wherein:
A is a radioactive entity which is a beta-energy emitter which produces Cherenkov radiation, said radioactive entity preferably being a radiochelate, namely a radiometal surrounded by a chelate or a radioelement which is non-metallic;
B is a fluorophore which absorbs electromagnetic radiation of a wavelength λ ranging from 300 nm to 500 nm;
C is a fluorophore which emits electromagnetic radiation of a wavelength λ ranging from 650 nm to 950 nm, and/or
C is a photosensitizer which produces reactive oxygen species ROSs;
D may be present or absent, and represents, when it is present, a vector entity, said vector entity preferably being a biomolecule or a nanoparticle vector,
L1, L2, L3 are each, independently of one another, an at least divalent linking group or a covalent bond, with the condition that

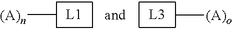

are not simultaneously present, which means that,

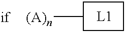

is present, then

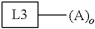

is absent and vice versa,
L4 is present if D is present and represents, when it is present, an at least divalent linking group or a covalent bond;
n is an integer equal to 1, 2, 3, 4 or 5;
m is an integer equal to 1, 2, 3, 4, 5, 6, 7 or 8; and wherein:
A activates B by CRET-type energy transfer,
B transfers the energy received from A to C, by intramolecular FRET-type energy transfer or by TBET-type energy transfer.

“TBET” is the acronym for “Through-Bond-Energy-Transfer”.

FRET-type transfer is the energy transfer between two fluorophores, the emission spectrum of the first of which corresponds to the absorption spectrum of the second. This is a transfer which takes place in space.

TBET-type transfer means that the transfer does not take place in space, but through bonds.

The radioactive entity A is denoted in the remainder of the text by A. It can consist of a (non-metallic) radioelement alone or of a radioelement of radiometal type surrounded by a chelating agent: the radiometal+chelating agent assembly is called radiochelate.

The fluorophore B is denoted in the remainder of the text by B. B can also be called “antenna”.

The fluorophore and/or photosensitizer C is denoted in the remainder of the text by C. C can also be called “platform”.

The vector entity D is denoted in the remainder of the text by D.

The compounds of the invention can therefore have a variable number of A (which can range from 1 to 5) and/or of B (which can range from 1 to 8).

The A or A_{S} is (are) represented by (A)ₙ.

The B or B_{S} is (are) represented by (B)ₙ.

(A)ₙ is bonded to C or (A)ₙ is bonded to (B)ₙ.

(B)ₘ is always bonded to C and vice versa (C is always bonded to (B)ₘ).

D, if it is present, is always bonded to C.

When it is stated in the present application that L1, which bonds (A)ₙ to C, is a covalent bond, this means that (A)ₙ is directly bonded to C without the intermediate of a linking group (in other words an electron of A forms a bond with an electron of C).

Similarly, when L2, which bonds C to (B)ₘ, is a covalent bond, this means that (B)ₘ is directly bonded to C without the intermediate of a linking group.

The same is true for L3, which bonds (B)ₘ to (A)ₙ, and for L4, which bonds C to D.

When L1, which bonds (A)ₙ to C, is a linking group, the latter will be an at least divalent radical (namely divalent, trivalent, tetravalent, etc.) depending on the meaning of n.

Likewise, when L2, which bonds C to (B)ₘ, is a linking group, the latter will be an at least divalent radical (namely divalent, trivalent, tetravalent, etc.) depending on the meaning of m.

The same is true for L3, which bonds (B)ₘ to (A)ₙ.

When L4, which bonds C to D, is a linking group, the latter will be a divalent radical.

By virtue of the conformational structure of the compounds of the invention, it is always A which activates B then B which activates C, even in the case where A is bonded to C.

According to one embodiment of the invention, C is a photosensitizer which produces reactive oxygen species ROSs, and in particular singlet oxygen with a quantum yield (ϕ_{Δ}) greater than 5%, preferably greater than 30%.

When, in the compound of formula (I) described above,

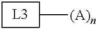

is absent, then it has the following structure (I-1):

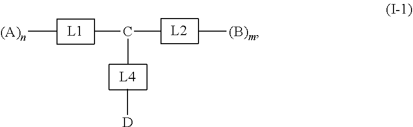

wherein A, B, C, D, L1, L2, L4, n and m are as defined above.

When L1, L2 and/or L4 are single covalent bonds, then (A)ₙ is directly linked to C, C is directly linked to (B)ₘ and/or C is directly linked to D.

By way of examples, if L1, L2 and L4 each represent a single covalent bond, the compound (I-1) is then simply represented by:

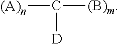

When D and L4 are absent, then the compound of formula (I-1) is simply represented by:

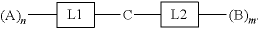

When, in the compound of formula (I) described above,

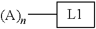

is absent, then it has the following structure (I-2):

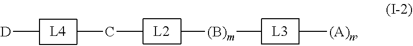

wherein A is a non-metallic radioelement and B, C, D, L2, L3, L4, n and m are as defined above.

When L3, L2 and/or L4 are single covalent bonds, then (A)ₙ is directly linked to (B)ₘ, (B)ₘ is directly linked to C and/or C is directly linked to D.

By way of examples, if L3, L2 and L4 are each a single covalent bond, the compound of the invention (I-2) is then simply represented by:

D-C—(B)ₘ-(A)ₙ.

When D and L4 are absent, then the compound of formula (I-2) above is simply represented by:

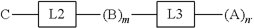

By way of examples, when n=m=1, D is absent and L1 and L2 are each a covalent bond, then the compound (I) of the invention, and more particularly (I-1), can simply be represented by:

A-C-B.

If there is an L1 between A and C which is a linking group, then the compound above is represented by:

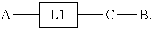

If D is present and L1, L2 and L4 each represent a covalent bond, then the compound (I-1) is represented by:

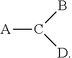

If there are linking groups L1 and L4, then the compound is represented by:

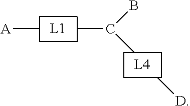

When n=m=1, D is absent and L3 and L2 are each a covalent bond, then the compound (I) of the invention, and more particularly (I-2), can simply be represented by:

A-B-C.

If there is an L3 between A and B which is a linking group, then the compound above is represented by:

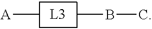

When n=m=1, D is present and L4, L2 and L3 each represent a single covalent bond, then the compound (I-1) is represented by:

A-B-C-D.

If there are linking groups L3, L2 and L4, then the compound is represented by:

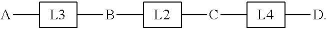

Again by way of examples, if n=5, m=1, L1 is a linking group, L2 is a covalent bond and D is absent, the compound (I) of the invention, and more particularly (I-1), is represented by:

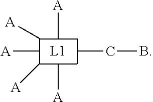

Still by way of examples, if m=4, n=1, L2 is a linking group, L1 is a covalent bond and D is absent, the compound of the invention (I-1) can be represented by:

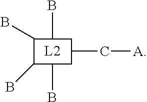

If D is present and L4 is a linking group, then the compound above is represented by:

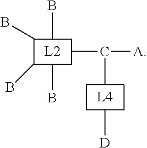

If m=4, n=1, L2 and L1 are linking groups and D is absent, the compound (I-1) can also be represented by:

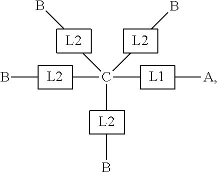

or by:

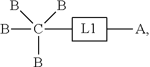

if L2 is a single covalent bond.

The examples given above are not of course exhaustive.

Moreover, the formulae exemplified are merely schemes intended to illustrate and to comprise the structure of the compounds of the invention. These schemes are not representative of the conformational structures of the compounds of the invention.

For the purposes of the invention, the term “radioactive entity” without other information can denote both a radiochelate and a non-metallic radioelement.

When A is a radiochelate, then A is always bonded to C.

When A is a non-metallic radioelement, then A can indifferently be bonded to C or to B.

In the compounds of formula (I-1), A is always bonded to C and A can indifferently represent a radiochelate or a non-metallic radioelement.

In the compounds of formula (I-2), A is always bonded to B and A then represents a non-metallic radioelement.

According to one advantageous embodiment of the invention, A is:
a radiochelate, the radiometal of which is chosen from the group comprising ⁹¹Y, ¹⁷⁷Lu, ⁶⁸Ga, ⁸⁹Zr, ⁶⁴Cu, ⁸⁹Sr, ²¹²Bi, ²¹³Bi, ⁴⁴Sc, ²²⁵Ac and ⁴⁴Sc and the chelating agent of which is chosen from the group comprising DOTAGA, DOTA, NOTA, NODAGA and DFO;
a non-metallic radioelement chosen from the group comprising ¹⁸F, ¹³¹I, ¹²⁴I and ³²P.

DOTAGA means 2,2′,2″-(10-(2,6-dioxotetrahydro-2H-pyran-3-yl)-1,4,7,10-tetraazacyclododecane-1,4,7-triyl)triacetic acid.

DOTA means 1,4,7,10-tetraazacyclododecane tetraacetic acid.

NOTA means 4,7-triazacyclononane-N,N′,N″-triacetic acid.

NODAGA means 2-(4,7-bis(carboxymethyl)-1,4,7-triazonan-1-yl)pentanedioic acid.

DFO means N¹-(5-aminopentyl)-N¹-hydroxy-N⁴-(5-(4-((5-(N-hydroxyacetamido)pentyl)amino)-4-oxobutanamido)pentyl)-succinamide.

An example of a radiochelate is the radiometal ⁹⁰Y chelated with the chelating agent DOTAGA or DOTA. They are respectively represented by “[⁹⁰Y]-DOTAGA” and “[⁹⁰Y]-DOTA”.

The radioactive entity [⁹⁰Y]-DOTA within the compound of formula (I) can be represented by the following radical:

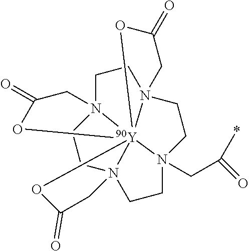

The radioactive entity [⁹⁰Y]-DOTAGA within the compound of formula (I) can be represented by the following radical:

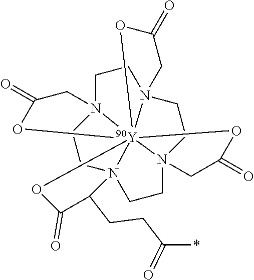

As already indicated, the radioactive entity A is a beta-energy emitter which produces CR. The compounds of the invention do not therefore need to be activated by an exterior radiation source, since they produce the CR by themselves, continuously, until the decay of A.

The half-life times of the radioactive entities A are very variable and can range from 68 minutes to 6.6 days.

By way of examples, the half-life time:
of the [⁹⁰Y]-DOTA or [⁹⁰Y]-DOTAGA radiochelate is 64.1 hours,
of ¹⁸F is 109.7 minutes,
of the [68Ga]-NOTA or [68Ga]-NODAGA radiochelate is 68 minutes,
of [⁸⁹Zr]-DFO is 78.4 hours,
of [¹⁷⁷Lu]-DOTA or [¹⁷⁷Lu]-DOTAGA is 6.64 days.

According to one embodiment of the invention, B is chosen from the group comprising a nucleus of the type, coumarin; substituted coumarin, in particular substituted with one or more hydroxyls and/or with a pyridinium, itself optionally substituted; pyranine; pyrene; BODIPY; substituted BODIPY, in particular phenyl-BODIPY, hydroxyphenyl-BODIPY, aza-BODIPY; fluorescin; rhodamine, in particular rhodamine 6G, rhodamine 101, rhodamine B, rhodamine 123; eosin, in particular eosin B, eosin Y; tryptophan, and mixtures thereof.

BODIPY is the abbreviation of boron-dipyromethene.

By way of example of coumarin substituted with a hydroxyl, mention may be made of hydroxycoumarin, substituted with two hydroxyls; mention may be made of dihydroxycoumarin.

By way of example of hydroxycoumarin substituted with a pyridinium, mention may be made of 4-methylpyridinium 7-hydroxycoumarin or pyridinium 4-propylsulfonate 7-hydroxycoumarin.

According to the invention, B can also comprise at least one solubilizing group, in particular chosen from the group comprising a sulfonate (SO₃⁻); a carboxylate (COO−); an ammonium (NR₄⁺) with R═H, alkyl or aryl; a phosphonate (PO₃²⁻); a pyridinium, which is preferably substituted; an imidazolium, and mixtures thereof.

By way of example, B can be a nucleus of pyrene type comprising at least one solubilizing group of formula NaSO₃, and preferably three NaSO₃ groups.

B can be a nucleus of the type coumarin or hydroxycoumarin substituted with a methylpyridinium or a pyridinium propylsulfonate.

According to one advantageous embodiment of the invention, when B is greater than 1, then the B_{S} may independently be identical or different within the compound (I) of the invention.

Having different B_{S} within one and the same compound makes it possible to cover a larger absorption window.

By way of example of such an embodiment, when n=2, then a B may represent a nucleus of coumarin or substituted coumarin type, and the other B a nucleus of BODIPY or substituted BODIPY type.

According to yet another advantageous embodiment of the invention, in the compounds of formula (I-2) wherein A represents a non-metallic radioelement, the latter may substitute for a part of B. In other words, a part of B can be replaced with A, which then means that A is therefore an integral part of B.

In this case, L3, which bonds A to B, never represents a linking group but always a covalent bond (A replaces a part of B).

By way of example of such an embodiment, when B is a nucleus of BODIPY or substituted BODIPY type, one of the two fluorines naturally present in BODIPY can be replaced with the non-metallic radioelement ¹⁸F.

In the invention, the term a nucleus “of X type” is intended to mean a nucleus “originating from a compound X”.

More specifically, once the compound X is involved in a bond with one or more other compounds, it will no longer be the compound X as such, but a compound originating from X or a compound of X type.

This compound of X type is the compound X as it is once involved in one or more bonds.

By way of example, in a compound of formula (I-1) wherein D is absent, when B is bonded to C, and B is a nucleus of hydroxycoumarin type, for example of 7-hydroxycoumarin type, it may be represented by the radical:

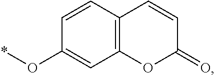 [SMILES: *Oc1ccc2ccc(=O)oc2c1]

whereas 7-hydroxycoumarin as such is represented by:

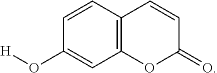 [SMILES: O=c1ccc2ccc(O)cc2o1]

Still by way of example, in a compound of formula (I-2) wherein D is absent, when B is bonded to C but also to A, and when B is a nucleus of dihydroxycoumarin type, for example of 4,7-hydroxycoumarin type, it may be represented by the divalent radical:

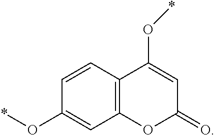 [SMILES: *Oc1ccc2c(O*)cc(=O)oc2c1]

Again by way of example, in a compound of formula (I-1) wherein D is absent, when B is bonded to C, and when B is a nucleus of pyranine type, it may be represented by the monovalent radical:

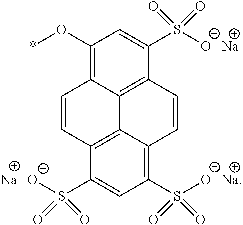 [SMILES: [Na+] ; *Oc1cc(S(=O)(=O)[O-])c2ccc3c(S(=O)(=O)[O-])cc(S(=O)(=O)[O-])c4ccc1c2c43 ; [Na+]]

In a compound of formula (I-2) wherein D is absent, when B is bonded to C but also to A, and when B is a nucleus of 8-phenyl-BODIPY type, it may be represented by the divalent radical:

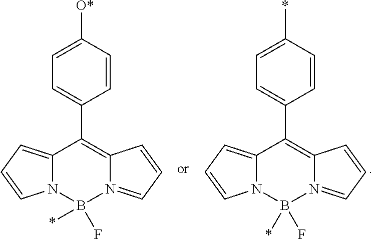

By way of more particular examples of B, mention may be made of a nucleus of coumarin, hydroxycoumarin, dihydroxycoumarin, methylpyridinium hydroxycoumarin, pyridinium propylsulfonate hydroxycoumarin, pyranine, pyrene, BODIPY, phenyl-BODIPY, hydroxyphenyl-BODIPY type.

According to another advantageous embodiment of the invention, C is chosen from the group comprising a nucleus of cyanine type, in particular cyanine-7, cyanine-5, cyanine-3; phthalocyanine, in particular silicon, zinc, magnesium, phosphorus, aluminum, indium phthalocyanine; naphthalocyanine, in particular zinc, magnesium, phosphorus, aluminum, silicon, indium naphthalocyanine; chlorin, in particular zinc, magnesium, phosphorus, aluminum, silicon, indium chlorin; bacteriochlorin, in particular zinc, magnesium, phosphorus, aluminum, silicon, indium bacteriochlorin.

According to the invention, C can also comprise at least one solubilizing group, in particular chosen from the group comprising a sulfonate (SO₃⁻); a carboxylate (COO−); an ammonium (NR₄⁺) with R═H, alkyl or aryl; a phosphonate (PO₃²⁻); a pyridinium, which is preferably substituted; an imidazolium, and mixtures thereof.

By way of examples, C can be a silicon phthalocyanine substituted with a pyridinium which is itself substituted (for example with a methyl or a propylsulfonate at the nitrogen atom of the pyridinium).

According to another advantageous embodiment of the invention, C is a nucleus of cyanine type. The cyanines are the name given to a family belonging to the polymethine group. They have many applications as fluorescent labels. The cyanines used in the context of the invention absorb mainly above 600 nm.

By way of example, in a compound of formula (I-1) wherein D is absent, n=1 (a single A), the nucleus C of cyanine type may be represented by the radical having the following general formula:

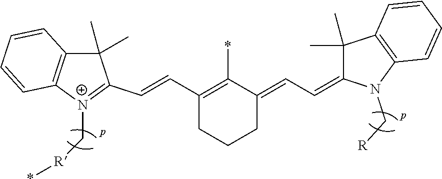

wherein:
p is an integer ranging from 0 to 4,
R′ represents CH₂, NH, N(alkyl), CO, NHCO, NHCOO, NHCOO(CH₂)ₚ, p=0 to 4,

R represents N₃, COOH, CH₃, NHCOO(alkyl),

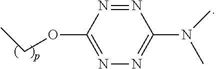

More particular examples of such nuclei of cyanine type are:

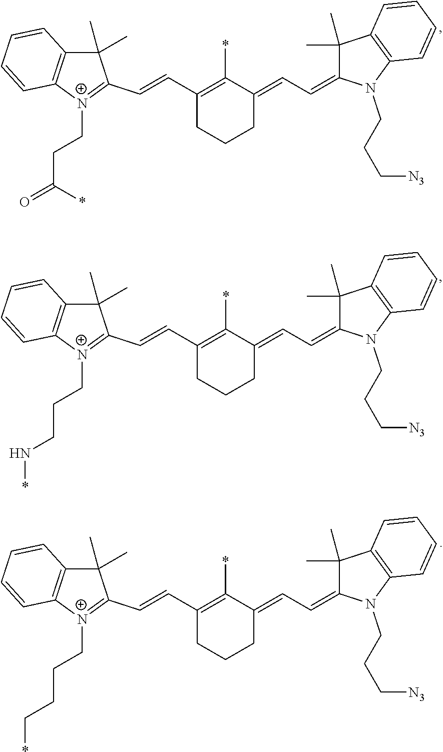 [SMILES: *C(=O)CC[N+]1=C(/C=C/C2=C(*)C(=C/C=C3/N(CCCN=[N+]=[N-])c4ccccc4C3(C)C)/CCC2)C(C)(C)c2ccccc21 ; *NCCC[N+]1=C(/C=C/C2=C(*)C(=C/C=C3/N(CCCN=[N+]=[N-])c4ccccc4C3(C)C)/CCC2)C(C)(C)c2ccccc21 ; *CCCC[N+]1=C(/C=C/C2=C(*)C(=C/C=C3/N(CCCN=[N+]=[N-])c4ccccc4C3(C)C)/CCC2)C(C)(C)c2ccccc21]

Still by way of example, in a compound of formula (I-1) wherein D is absent or present, n=1 (a single A) or greater than 1, the nucleus C of cyanine type may be represented by the radical having the following general structure:

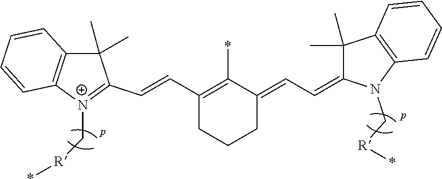

wherein:

p is an integer ranging from 0 to 4,

each R′ represents, independently of the other, CH₂, NH, N (alkyl), CO, NHCO, NHCOO, NHCOO(CH₂)ₚ.

More particular examples of such nuclei of cyanine type are:

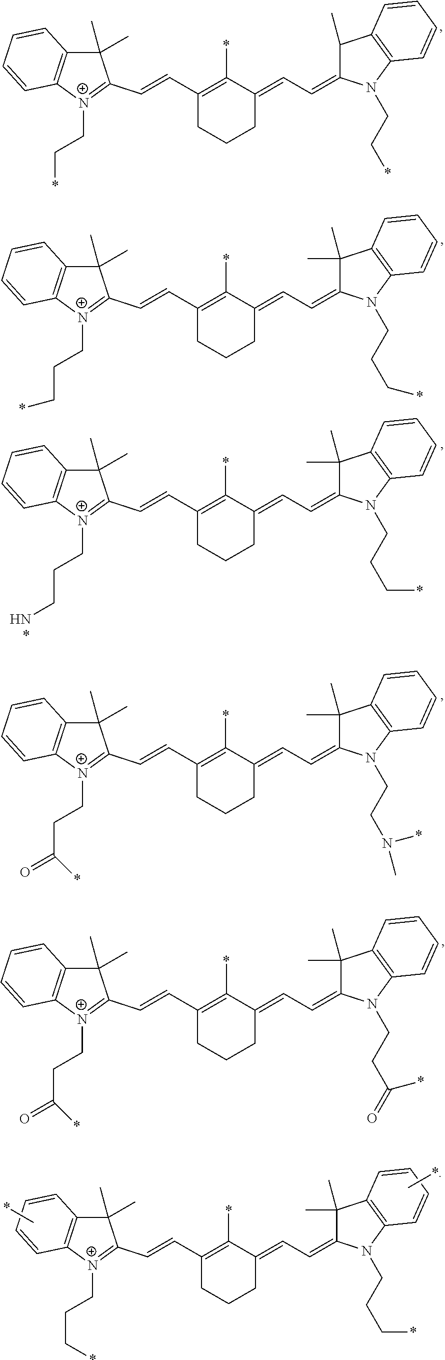

In a compound of formula (I-2) wherein D is absent, the nucleus C of cyanine type may be represented by the radical having the following general structure:

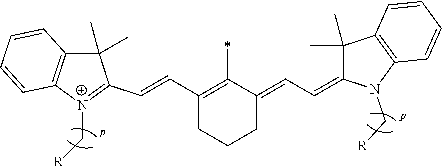

wherein:

p is an integer ranging from 0 to 4,

each R represents, independently of the other, N₃, COOH, CH₃, NHCOO(alkyl),

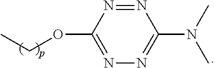

More particular examples of such nuclei of cyanine type are:

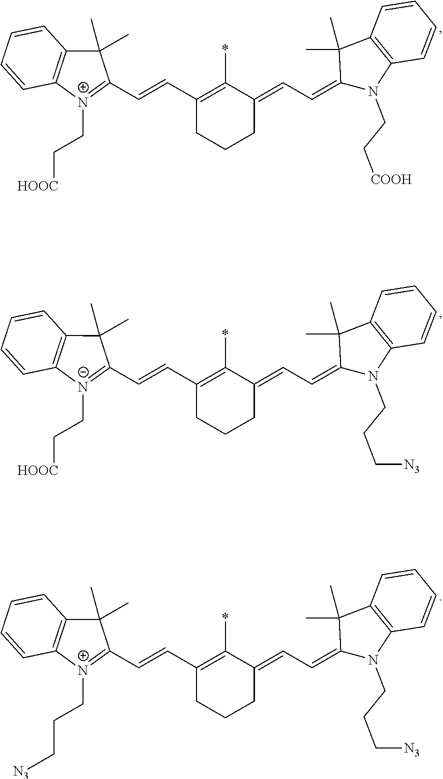

According to another advantageous embodiment of the invention, C is a nucleus of phthalocyanine type.

In a compound of formula (I-1), the nucleus C of phthalocyanine type may be represented by one of the multivalent radicals having the following formula:

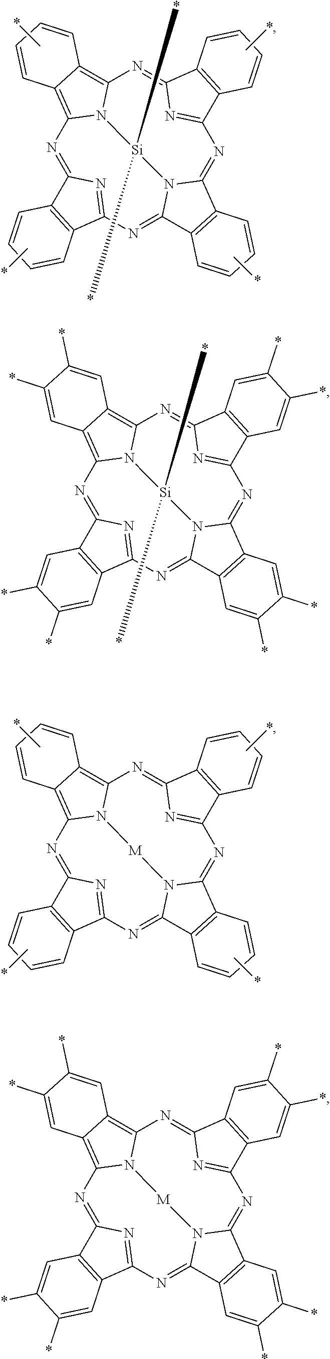

wherein M represents Zn (zinc), Mg (magnesium), P (phosphorus), Al (aluminum) or In (indium).

In addition to the solubilizing group (s) that can be borne by C, it is also possible, according to one advantageous embodiment of the invention, for C to bear one or more functional groups. These functional groups may in particular act as an “attachment function” for D.

Thus, when C is for example a nucleus of phthalocyanine type with a metal M at the center of the ring, then it will be possible to bond a functional group to the metal M, said functional group being intended to react with another functional group which is an integral part of a precursor of D (the precursor of D denoting D before it is bound to C) or else which is specially grafted to the precursor of D. The reaction between the two functional groups of C and of the precursor of D will form the linking group L4, which bonds C with D.

Examples of functional groups that can be bonded with the metal M of the phthalocyanine are:

—O—(CH₂)_{q}—N₃ with q an integer ranging from 1 to 4,

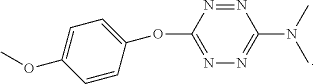

According to one advantageous embodiment of the invention, in the compounds of formula (I-1) or (I-2), when m=1 (a single B), the linking group L2, which bonds C and B, is:
a divalent radical —O—, —S—, —NH—, —N(alkyl);
a saturated or unsaturated, divalent hydrocarbon-based radical having from 1 to 4 carbon atoms, preferably 2 carbon atoms, in particular chosen from —C≡C—, —CH₂—C≡C—CH₂—, —CH═CH—, —CH₂—CH═CH—CH₂— or —(CH₂)_{q}—, q is an integer ranging from 1 to 4, preferably from 1 to 2.

O represents oxygen, S sulfur and N nitrogen.

Again according to the invention, in the compounds of formula (I-1) or (I-2), when m=2, 3 or 4, L2 is a multivalent linking group which acts as a platform which makes it possible to collect the B_{S} and to bond them with C.

Examples of multivalent linking radicals L2 are:

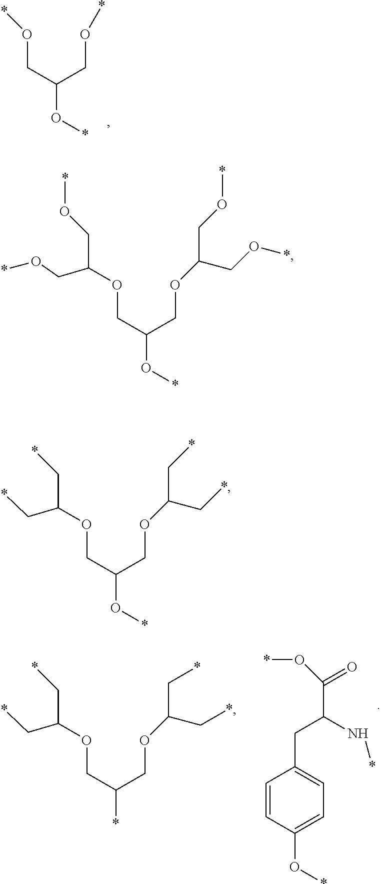

According to another embodiment of the invention, in the compounds (I-1) and/or (I-2), L2 is a covalent bond.

According to yet another advantageous embodiment of the invention, in a compound of formula (I-1), the linking group L1, which bonds A to C, can comprise a function of amide, carbonyl, amine, triazole, pyridazine, peptide, urea, thiourea, thioether or maleimide type.

Examples of multivalent linking radicals L1 are:

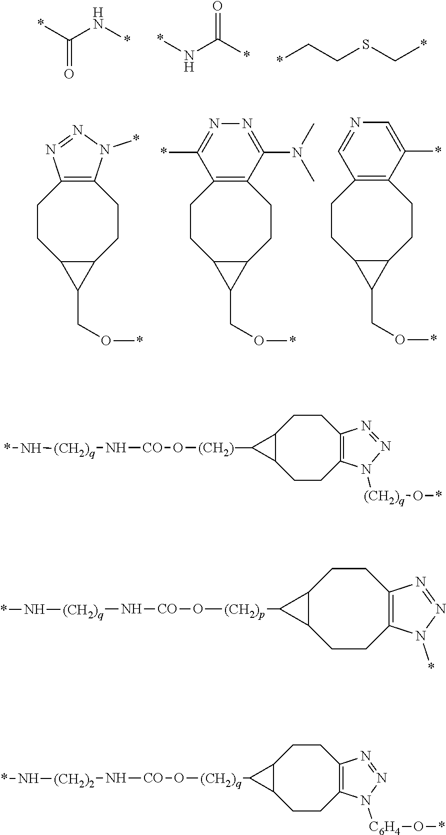

*—(CH₂)ₚ—CO—(CH₂)ₚ—*, *—NH—(CH₂)_{q}—NH—*, *—NH—C₆H₄—O—(CH₂)ₚ—*, *—(CH₂)_{q}—CO—NH—(CH₂)_{q}—*, with q=1 to 4 and p=0 to 4.

According to the invention, in a compound of formula (I-1), when n=2, 3, 4 or 5, A=radiochelate, L1 is a multivalent linking group which acts as a platform which makes it possible to collect all the As and then to make a bond with C.

By way of example of such a multivalent compound, mention may be made of the radical 1,2,3,4,5,6-benzenehexamethanamine which makes it possible to bear up to five radiochelates A:

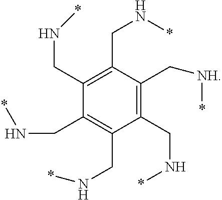 [SMILES: *NCc1c(CN*)c(CN*)c(CN*)c(CN*)c1CN*]

When it bears the five As, it is represented by the radical of formula:

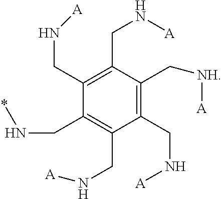

According to another embodiment of the invention, in the compounds (I-1), L1 is a covalent bond.

According to yet another advantageous embodiment of the invention, in the compounds of formula (I-2), when n=m=1, A is a non-metallic radioelement, the linking group L3, which bonds A and B, is:

—(CH₂)_{q}—O—(CH₂)_{q}—; —(CH₂)_{q}—O—; —(CH₂)_{q}—O—; —(CH₂)_{q}—S—(CH₂)_{q}—; —(CH₂)_{q}—S—(CH₂)_{q}—S—; —(CH₂)_{q}—NH—(CH₂)_{q}—; —(CH₂)_{q}—NH—(CH₂)_{q}—NH—; —(CH₂)_{q}—; q is an integer ranging from 1 to 4.

According to another embodiment of the invention, in the compounds (I-2), L3 is a single covalent bond.

According to another advantageous embodiment, the compound of the invention is of the formula (I-1) wherein A is a radiochelate bonded to C by means of a linking group L1, and C is bonded to B by means of L2 which is a covalent bond.

According to another particularly advantageous embodiment, the compound (I) of the invention comprises a vector entity D.

This vector entity can be a biomolecule such as a peptide; a protein; a protein of antibody type; a protein of antibody fragment type, such as Fab, Fab′2, Fab′, ScFv, nanobody, affibody, diabody; an aptamer.

According to one embodiment of the invention, the linking group L4, namely the group bonding D to C or vice versa, can comprise a function of amide, carbonyl, amine, triazole, pyridazine, peptide, urea, thiourea, thioether or maleimide type.

By way of examples, the linking group L4 can be represented by one of the following radicals:

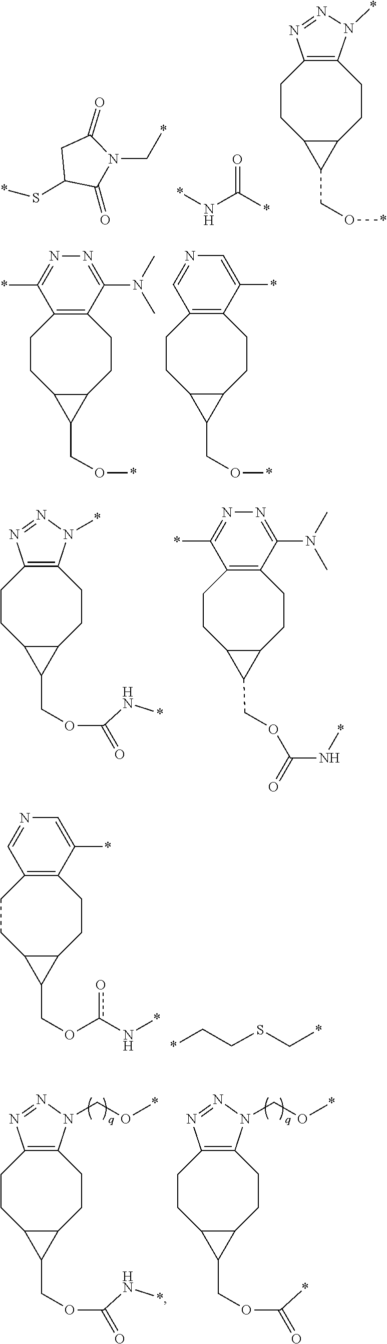

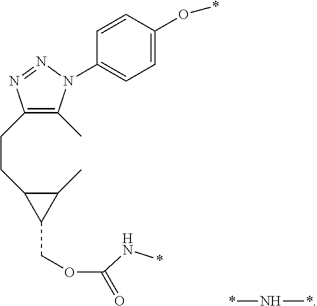

According to another embodiment of the invention, L4 is a single covalent bond in the compounds of formula (I) of the invention.

By way of example, in a compound of formula (I-1) of the invention, when n=m=1, A=radiochelate and when C is a nucleus of cyanine type bonded to D, then C can be represented by the trivalent radical having the following formula:

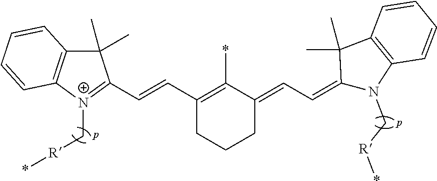

wherein:

p is an integer ranging from 0 to 4, each R′ represents, independently of the other, CH₂, NH, N(alkyl), CO, NHCO, NHCOO, NHCOO(CH₂)ₚ.

Another subject of the present invention lies in the method for preparing the compounds of the invention.

Initially, a first single-molecule structure is prepared which comprises C and from one to four Bs.

Next, up to five As can be grafted onto the conjugate thus formed, so as to form a new single-molecule structure.

Finally, D can be grafted onto this single-molecule structure, so as to re-form a new single-molecule structure.

Those skilled in the art will know which method to use in order to attach a specific molecule to a chosen substrate.

By way of example, the cyanines that will be used to prepare a compound of the invention of formula (I) wherein C is a nucleus of cyanine type will be symmetrical or asymmetrical cyanines having one of the following structures:

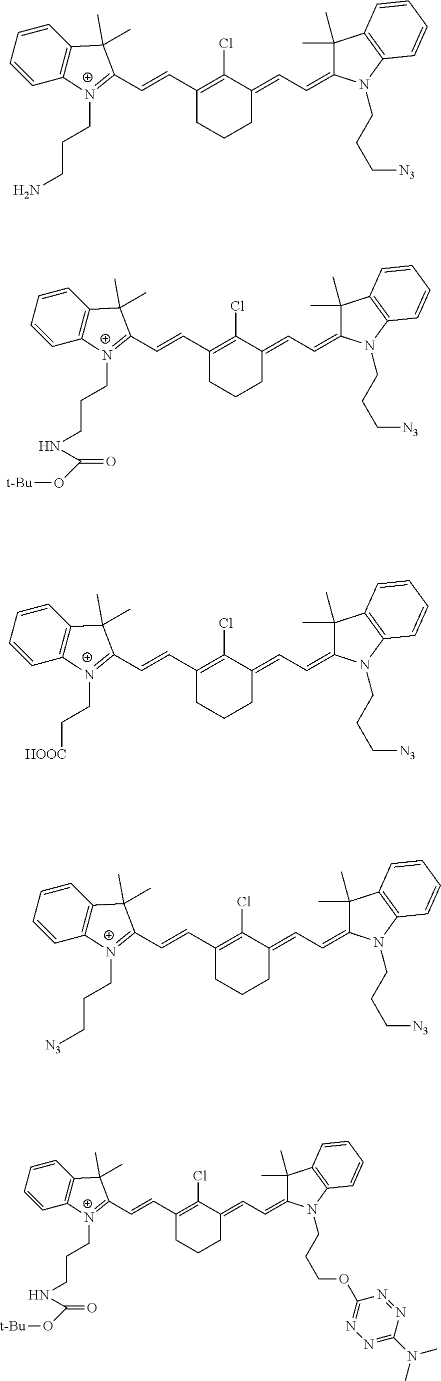

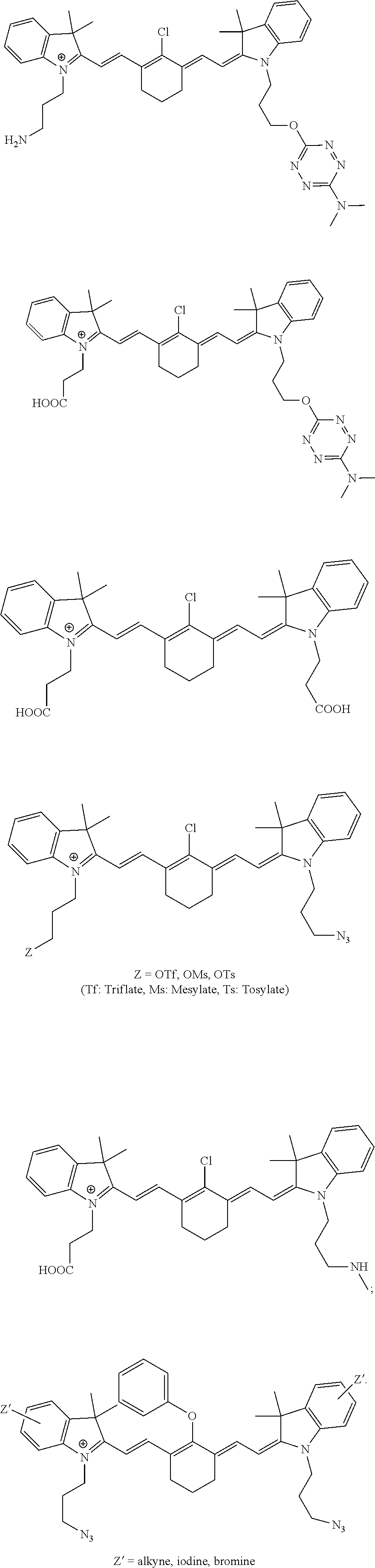

B may be grafted to the cyanine by substitution of the halogen (for example the chlorine as represented above) of the cyanine.

In a compound of formula (I-1), A will be grafted to the cyanine by means of the functional group NH₂, N₃, COOH, NHCOO(alkyl), etc.

The linking group L4, namely the group bonding D to C or vice versa, results from the reaction between (1/) a functional group of a precursor of D and (2/) a functional group borne by C before it is involved in a bond with D.

The term “precursor” of D is intended to mean the entity D before it is involved in a bond with C.

C thus advantageously comprises at least one functional group in order to be able to react with a functional group of the precursor molecule of D.

When the compound of the invention comprises a nucleus C of cyanine type, the functional group of the nucleus of cyanine type may for example be an azide (N₃), tetrazine, activated ester (which is an activated form of a carboxylic acid function) or triazine group.

Thus, L4, which is the group bonding D and C, may be a radical comprising:
a triazole function, which results from the reaction of an azide function of C with an alkyne function of the precursor of D,
a pyridazine function, which results from the reaction of a tetrazine function of C with a bicyclo-nonyne function of the precursor of D,
an amide function which results from the reaction of the activated form of a carboxylic acid function of C with an amine function of the precursor of D.

When the compound of the invention comprises a nucleus C of phthalocyanine type, the phthalocyanine is bonded to D by means of a functional group, comprising an azide function N₃, bonded to the metal M of the phthalocyanine.

In general, the methods used in the context of the invention are the general methods of organic synthesis, purifications by chromatography and LC-MS (liquid chromatography-mass spectrometry).

The syntheses are convergent (synthesis of the platform C, or even of certain antennas B) and each compound is characterized by a range of spectroscopic methods: proton and carbon NMR, high-resolution and low-resolution mass spectrometries, UV/Vis (visible) and infrared spectrometries.

The purity of the synthons and of the targets is determined by HPLC. At the outcome, the BC compounds bearing the entity suitable for radiolabeling are radiolabeled using the techniques of radiolabeling chemistry with dedicated protections on a dedicated site. The radiochemical purity is verified by radio-TLC and/or by radio-HPLC.

The compounds are then conjugated to a biomolecule, for example an antibody labeled with a bioorthogonal chemical function; the techniques for analysis and purification of the bioconjugates comprise MALDI-TOF and UV/Vis mass spectrometry and the purification is carried out on FPLC and Sephadex size exclusion columns.

The new compounds of the invention can advantageously be used:
for near-IR Cherenkov luminescence imaging when C is a fluorophore,
for deep biological tissue treatment by Cherenkov photodynamic therapy when C is a photosensitizer,
for both when C is a fluorophore and a photosensitizer.

A subject of the invention is thus also the use of a compound of formula (I) as defined above, for an application for near-infrared Cherenkov luminescence imaging.

Compounds numbers 1, 3, 5-7, 11-18, 21 and 22 of table 1 are particularly advantageous for an application for near-IR CLI.

The invention also relates to a method of diagnosis by near-IR Cherenkov luminescence imaging, said method being characterized in that it comprises the administration to a subject of a compound of formula (I), said compound preferably comprising D.

A subject of the invention is also a compound of formula (I) as defined above, for use for the treatment of deep biological tissues by Cherenkov photodynamic therapy.

Another subject of the invention lies in a compound of formula (I) as defined above, for use for the treatment of deep biological tissues by Cherenkov photodynamic therapy, said Cherenkov photodynamic therapy being used in combination with at least one other anticancer treatment.

This is because the stress induced by the Cherenkov-PDT effect on the deep tumor can induce cell mortality by the direct PDT therapeutic effect. When small amounts of compounds of the invention are used, the stress induced by the Cherenkov-PDT effect can have a non-lethal effect which, however, makes it possible to weaken the tumor tissues and to thus make them more sensitive to other therapies. Thus, a first step of treatment of the tumor by the action of Cherenkov-PDT using the compounds of the invention makes it possible to potentiate the action of one or more other therapies to be carried out in a second step.

Compounds numbers 2, 4, 8-10, 19 and 20 of table 1 are particularly advantageous for use for the treatment of deep biological tissues by Cherenkov PDT.

In addition, these compounds 2, 4, 8-10, 19 and 20 are also advantageous for an application for near-IR CLI.

The invention also relates to a method for treating cancer by Cherenkov photodynamic therapy, and in particular a method for treating deep biological tissues, said method being characterized in that it comprises the administration in a subject of a compound of formula (I) as defined above, said compound preferably comprising D.

The method for treating cancer by Cherenkov photodynamic therapy as described above can also be combined with at least one other anticancer treatment method.

The luminescence optical properties of the compounds of the invention and of the BC intermediates are examined by conventional spectrofluorimetry (laser source) and, in the case of the radiolabeled compounds, by spectrofluorimetry in the presence of radioelements in bioluminescence mode, and an optical imager.

The photosensitizing properties of the compounds dedicated to Cherenkov-PDT are examined by UV/Vis spectrometry by monitoring the decrease in the DPBF (diphenylbenzofuran) absorption band, but also by cellular tests and studying the cytotoxicity.

Finally, the in vivo studies are carried out on xenografted mice bearing cancer models, which are chosen as being superficial cancers in the case of the CLI studies, or else as being deep cancers in the case of the Cherenkov-PDT studies.

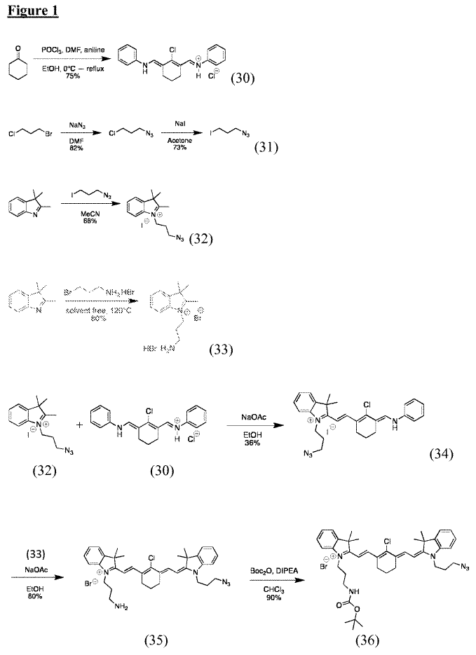

FIG. 1 is a scheme of the synthesis of asymmetric cyanines (35) and (36) which will be used to prepare compounds (I) of the invention wherein C is a nucleus of asymmetric cyanine type.

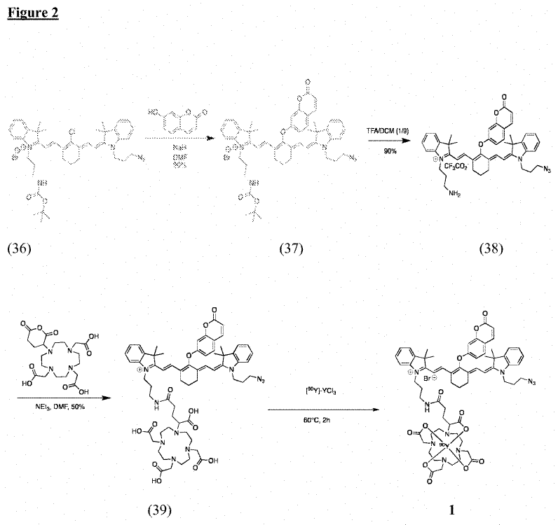

FIG. 2 is a scheme of the synthesis of the compound 1 of the invention.

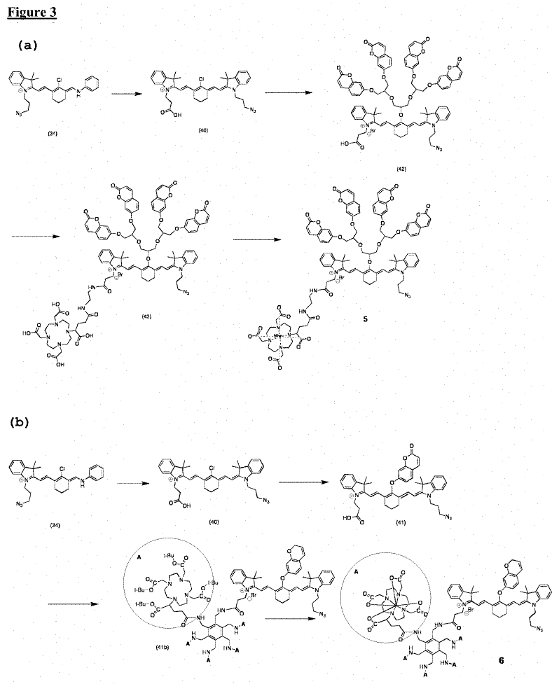

FIG. 3 is a scheme of the synthesis of the compounds 5 (FIG. 3a) and 6 (FIG. 3b) of the invention.

FIG. 4 is a scheme of the synthesis of the compounds 21 (FIG. 4a) and 11 (FIG. 4b) of the invention.

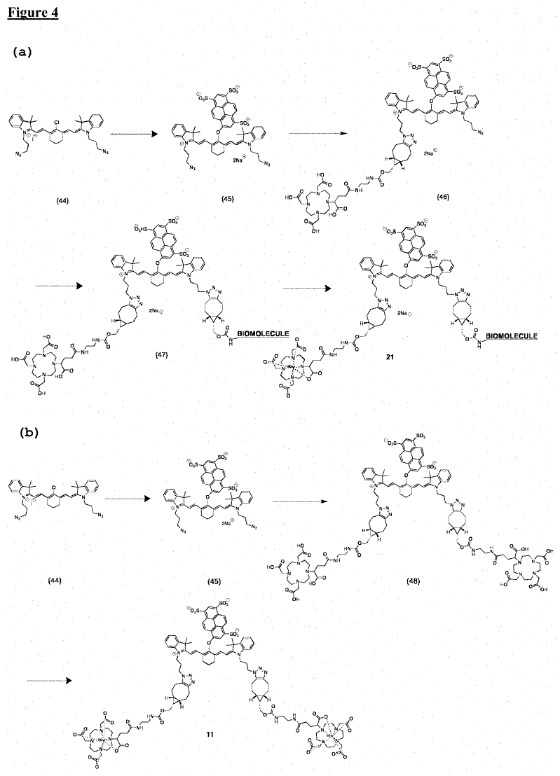

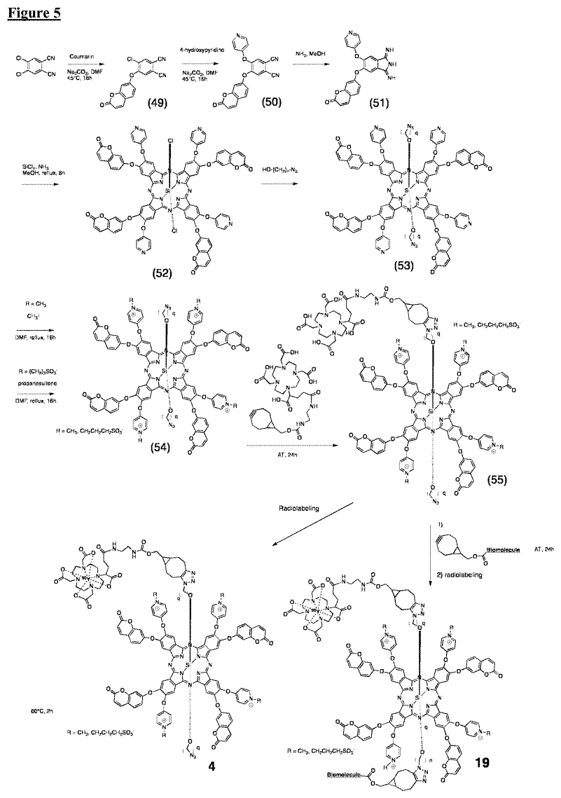

FIG. 5 is a scheme of the synthesis of the compounds 4 and 19 of the invention.

The following examples illustrate the invention; they do not limit it in any way.

### EXAMPLE 1

Preparation of Compounds (I) of the Invention

The methods for preparing several compounds which are subjects of the invention are described in detail in this example.

Separations and Analyses by HPLC

System A: HPLC-MS (Hypersil C18 column, 2.6 μm, 2.1×50 mm) with H₂O 0.1% formic acid (FA) as eluent A and CH₃CN 0.1% FA as eluent B [linear gradient from 5 to 100% of B (5 min) and 100% of B (1.5 min)] at a flow rate of 0.5 ml/min. The UV detection is carried out at 650 and 750 nm.

System B: HPLC (Hypersil C18 column, 5 μm, 10×250 mm) with H₂O 0.1% FA as eluent A and CH₃CN 0.1% FA as eluent B [linear gradient from 20 to 60% of B over the course of 40 min] at a flow rate of 3.5 ml/min. The UV detection is carried out at 700 and 780 nm.

### 1/ Synthesis of Asymmetric Cyanines of Formulae (35) and (36) (see FIG. 1)

In the compounds No. 1, 3, 5-7 and 12-17 of the invention, C is a nucleus of asymmetric cyanine type.

The method of synthesis of the asymmetric cyanines (35) and (36) used for preparing the compounds of the invention has no precedent and was developed by the inventors. It is therefore described in detail below (see also FIG. 1).

### Synthesis of the Compound (30)

Phosphorus oxychloride (5.6 ml, 60 mmol) is added dropwise at 0° C. to anhydrous DMF (6.5 ml, 84 mmol). After 30 min, cyclohexanone (2.75 ml, 27 mmol) is then added, resulting in a change in color of the reaction mixture which becomes orange and which is brought to reflux for 1 h in a water bath. After having cooled the mixture to ambient temperature, an aniline/ethanol mixture [1:1 (v/v), 90 ml] is added dropwise. An exothermic reaction, generation of HCl and solidification follow on from this. After addition of aniline, the reaction mixture which is deep purple in color is poured into an ice-cold water/concentrated HCl mixture [10:1 (v/v) 80 ml]. Crystals form in the solution stored at 4° C. for 12 h. After filtration, the crystals are washed with cold water and then diethyl ether and dried to give 7.19 g (75%) of the product (30).

### Synthesis of (31)

Sodium azide (650 mg, 10 mmol) is added to a solution of 1-bromo-3-chloropropane (1.57 g, 10 mmol) in 15 ml of DMF (N,N-dimethylformamide). After stirring for 5 h at ambient temperature, the reaction mixture is poured into 80 ml of water and extracted with ether (3×50 ml). The organic phases are combined and washed with water (2×50 ml) and brine (100 ml), then dried over MgSO₄ and finally concentrated under reduced pressure. Added to the residue obtained (0.98 g, 8.23 mmol), which is redissolved in acetone (50 ml), is sodium iodide (2.47 g, 16.47 mmol). The resulting mixture is brought to reflux with stirring for 16 h, then is subsequently poured into 50 ml of water and extracted with ethyl acetate (3×50 ml). The organic phases are washed with water (2×50 ml), dried over MgSO₄ and concentrated under reduced pressure to give 1.27 g (60%) of the product (31), which is in the form of a yellow oil. No purification is necessary. ¹H NMR (500 MHz, CDCl₃, 300 K): δ (ppm)=2.03 (m, 2H); 3.25 (t, J=6.6 Hz, 2H); 3.43 (t, J=6.6 Hz, 2H). ¹³C NMR (125 MHz, CDCl₃, 300 K) δ (ppm)=2.42; 32.46; 51.59.

### Synthesis of (32)

A solution of 2,3,3-trimethylindolenine (377 mg, 2.37 mmol) and of azido-3-iodopropane (31) (1 g, 4.74 mmol) in acetonitrile is brought to reflux for 2 days. The color of the solution changes from pale orange to dark green. The solvent is evaporated off under reduced pressure and 5 ml of dichloromethane are then added. This solution was added dropwise to diethyl ether (50 ml) resulting in the precipitation of a dark green compound. The solid is recovered by filtration, then 5 ml of dichloromethane are added and the process is repeated twice. The solid is vacuum-dried to give 596 mg (68%) of the product (32) which is in the form of a dark green to brown solid.

¹H NMR (500 MHz, CDCl₃, 300 K): δ (ppm)=1.65 (s, 6H); 2.32 (m, 2H); 3.19 (s, 3H), 3.70-3.77 (m, 2H); 4.91 (t, J=7.2 Hz, 2H); 7.52-7.64 (m, 3H); 7.84-7.89 (m, 1H). ¹³C NMR (125 MHz, CDCl₃, 300 K): δ (ppm)=196.67; 141.62; 141.08; 130.30; 129.80; 123.40; 115.87; 54.85; 49.04; 47.87; 27.44; 23.26; 17.50.

### Synthesis of (33)

A mixture of 2,3,3-trimethylindolenine (331 mg, 2.08 mmol) and of 3-bromopropylamine hydrobromide (456 mg, 2.08 mmol) is heated at 120° C. in a sealed tube for 10 h. The solid residue formed is cooled and washed abundantly with diethyl ether then a mixture of Et₂O:CHCl₃ (1:1) to give 574 mg (74%) of the product (33).

¹³C NMR (125 MHz, MeOD, 300 K): δ (ppm)=199.28; 143.38; 142.32; 131.36; 130.60; 124.80; 116.40; 56.19; 46.46; 37.86; 29.79; 16.81; 22.85.

### Synthesis of (34)

The compound (32) (300 mg, 0.81 mmol) and sodium acetate (70 mg, 0.85 mmol) are dissolved in 30 ml of dry ethanol, resulting in a green solution. The compound (30) (313 mg, 0.97 mmol) is then added with 10 ml of dry ethanol, resulting in a purple/blue solution. The reaction mixture is brought to reflux for 2 h and the progression of the reaction is monitored by LC-MS (liquid chromatography-mass spectrometry). Half the volume of solvent is distilled under reduced pressure and the more concentrated reaction mixture is poured into 70 ml of Et₂O. The solid is washed with Et₂O (3×50 ml) and vacuum-dried. The solid is then purified with a chromatographic column on silica gel (DCM/MeOH 98/2 vol.) to give 175 mg (36%) of the pure product (34). It should be noted that the color of the compound depends on its state of protonation; it appears blue in TLC because of the acidity of the silica.

¹H NMR (500 MHz, CDCl₃, 300 K): δ (ppm)=1.66 (s, 6H); 1.87 (p, J=6.2 Hz, 2H); 1.93-2.08 (m, 2H); 2.61-2.67 (m, 2H); 2.79 (t, J=6.1 Hz, 2H); 3.42 (t, J=6.2 Hz, 2H); 3.79 (m, 2H); 5.57 (d, J=12.6 Hz, 1H); 6.70 (d, J=7.8 Hz, 1H); 6.92 (t, J=7.4 Hz, 1H); 7.14-7.23 (m, 5H); 7.37 (m, 3H); 7.62 (d, J=12.6 Hz, 1H); 8.84 (s, 1H). ¹³C NMR (125 MHz, CDCl₃, 300 K): δ (ppm)=159.86; 158.13; 129.50; 129.40; 129.30; 128.17; 125.77; 122.12; 121.36; 121.23; 121.19; 120.82; 106.69; 93.40; 77.51; 77.26; 77.01; 66.10; 49.03; 46.49; 39.67; 36.09; 29.95; 28.61; 26.94; 26.20; 21.60; 15.52.

### Synthesis of the Cyanine (35) (Fluorophore)

The compounds (34) (105 mg, 0.175 mmol), (33) (119 mg, 0.315 mmol) and sodium acetate (26 mg, 0.315 mmol) are dissolved in 10 ml of dry ethanol to give a green solution. The mixture is brought to reflux with stirring for 7 h and its color changes to dark blue. The progression of the reaction is monitored by UV-Visible and LCMS. The reaction mixture is concentrated under reduced pressure and then poured into 30 ml of Et₂O. The resulting solution is filtered to give a brownish solid which is washed with Et₂O and purified on a size exclusion column using chloroform as eluent, to give 300 mg (40%) of a pure product (35) which is in the form of a green solid. ¹H NMR (600 MHz, DMSO, 323 K): δ (ppm)=1.70 (s, 6H); 1.70 (s, 6H); 1.86-1.91 (m, 2H); 2.01-2.07 (m, 4H); 2.74-2.77 (m, 4H); 2.99 (m, 2H); 3.53 (t, J=6.5 Hz, 2H); 4.32 (q, J=7.0 Hz, 4H); 6.32 (d, J=13.9 Hz, 1H); 6.41 (d, J=14.3 Hz, 1H); 7.27-7.67 (m, 8H); 7.87 (s, 2H); 8.27 (t, J=14.0 Hz, 1H); 8.32 (d, J=14.2 Hz, 1H). Mass spectrum: m/z=595.3311 [M-2Br]⁻ (calculated for C₃₆H₄₅Br₂ClN₆: 754.1751).

### Synthesis of the Cyanine (36) (Fluorophore)

The compound (35) (300 mg, 0.396 mmol), di-tert-butyl dicarbonate (130 mg, 0.594 mmol) and DIPEA (N,N-diisopropylethylamine) (255 mg, 1.98 mmol) are dissolved in 15 ml of chloroform to give a green solution. The mixture is brought to reflux with stirring, and the progression of the reaction is monitored by LCMS. After cooling to ambient temperature, the crude mixture is washed with water (2×40 ml) and with a 0.2 M solution of hydrochloric acid (30 ml). The organic phases are combined, and concentrated under reduced pressure, and then the compound is isolated and purified with a size exclusion column using chloroform as eluent, to give the pure product (36) which is in the form of a green solid. Mass spectrum: m/z=695.5 [M−Br]⁻ (calculated for C₄₁H₅₂BrClN₆O₂: 774.30). HPLC, retention time: 5.95 min. UV-Vis: 777 nm.

### 2/ Synthesis of the Compound 1 (See FIG. 2)

### Synthesis of the Coumarin-Cyanine Conjugate (37)

7-Hydroxycoumarin (4.1 mg, 0.026 mmol) and sodium hydride (2.0 mg, 0.0515 mmol) are dissolved in 1 ml of DMF and the mixture is stirred at ambient temperature for 10 min. The cyanine (36) (10 mg, 0.0128 mmol) is subsequently added and then the progression of the reaction is monitored by LCMS. After approximately 20 minutes, the DMF is distilled under reduced pressure, and the product is taken up with CHCl₃ then washed with water and purified on a small plug of silica gel (solvent: DCM). The compound (37) is obtained in the form of a green solid (10 mg, 90%). Mass spectrum: m/z=821.4 [M−Br]⁻ (calculated for C₅₀H₅₇BrN₆O₅: 900.35). HPLC, retention time: 5.6 min. UV-Vis: 307, 777 nm.

### Synthesis of the Compound (38)

The compound (37) (10 mg, 0.013 mmol) is dissolved in 2 ml of a TFA/DCM mixture (1/9 vol.) and the resulting solution is stirred for 1 h at ambient temperature. 10 ml of DCM are added to this mixture, and the organic phase is subsequently washed with a saturated solution of NaHCO₃ (2×25 ml), then dried with MgSO₄, then concentrated under reduced pressure to obtain 9 mg (90%) of product (38). Mass spectrum: m/z=721.4 [M−CF₃CO₂⁻]⁻ (calculated for C₄₅H₄₉N₆O₃: 721.92). HPLC, retention time: 4.4 min. UV-Vis: 307, 777 nm.

### Synthesis of the Compound (39)

DOTA-GA anhydride (2,2′,2″-(10-(2,6-dioxotetrahydro-2H-pyran-3-yl)-1,4,7,10-tetraazacyclododecane-1,4,7-triyl)triacetic acid) (9.1 mg, 0.0198 mmol) and triethylamine (11 mg, 0.105 mmol) are added to a solution of the compound (38) (9 mg, 0.0107 mmol) in 1.5 ml of DMF, and then the resulting mixture is stirred for 24 h at 50° C. After the DMF has been evaporated off, the product is taken up in CHCl₃ and washed with water. The product is subsequently purified using a size exclusion column with CHCl₃ to give 7 mg (50%) of pure product (39). Mass spectrum: m/2z=590.2 (calculated for C₆₄H₇₉N₁₀O₁₂: 1180.39). HPLC, retention time: 4.3 min. UV-Vis: 307, 777 nm.

### Synthesis of the Compound 1 of the Invention

The precursor (39) is placed in an ammonium acetate buffer, pH 4.5, and placed in the presence of a radioactive source (⁹⁰YCl₃ source), so as to obtain the desired specific activity. The mixture is heated at 80° C. for 2 hours and the reaction is monitored by radio-ITLC. The compound 1 of the invention is obtained.

### 3/ Synthesis of the Compounds 7, 12 and 13 of the Invention

The compounds 7, 12 and 13 are synthesized according to a synthesis method comparable to that described for the compound 1, but with the following differences: the steps corresponding to the introduction of the radiochelate of ⁹⁰Y-[DOTAGA] type are not carried out.

Instead, the ¹⁸F-radiolabeling steps for the compounds 7 and 13 are carried out starting from the corresponding alcohol which is converted into a triflic ester which is then placed in the presence of a salt of Na¹⁸F type so as to produce substitution of the triflate with the ¹⁸F.

The synthesis of the compound 12 of the invention is carried out according to a method comparable to the compound 1 (introduction of a BODIPY derivative bearing a 4-hydroxyphenyl group in meso position). The BODIPY compound bearing two non-radioactive ¹⁹F fluorine atoms will react with DMAP (dimethylaminopyridine) by substitution of one of the two fluorine atoms, thus resulting in a BODIPY-DMAP adduct wherein the DMAP, now in quaternized form, is a good leaving group. In the presence of a salt of Na¹⁸F type, the ¹⁸F will substitute the quaternized DMAP, resulting in the ¹⁸F-radiolabeled moiety.

### 4/ Synthesis of the Compounds 5 and 6 of the Invention (See FIG. 3)

The compounds 5 and 6 can be obtained from an asymmetric cyanine (40) resulting in the compound (42) or (41) which are respectively the precursors of the compounds 5 (FIG. 3a) and 6 (FIG. 3b) of the invention.

### Synthesis of the Compound (40)

The compound (34) (170 mg, 0.28 mmol), the compound (31) (88 mg, 0.28 mmol) and sodium acetate (23 mg, 0.28 mmol) are dissolved in 12 ml of dry ethanol, giving a brown solution. The mixture is stirred and brought to reflux for 2 h, where it becomes dark green. At the end of UV-Visible and LCMS verifications, the reaction mixture is concentrated under reduced pressure and poured into 75 ml of Et₂O. The solution is filtered to give a brownish solid which is washed with Et₂O and purified by silica gel chromatography (using a DCM/MeOH solvent gradient of 98/2 vol. to 90/10 vol.) so as to give 75 mg of pure product (40) in the form of a dark green solid.

¹H NMR (500 MHz, CDCl₃, 300 K): δ (ppm)=1.9 (m, 12H); 1.90 (m, 2H); 2.07 (t, J=6.5 Hz, 2H); 2.56-3.08 (m, 6H); 3.53 (t, J=6.0 Hz, 2H); 4.14 (t, J=7.1 Hz, 2H); 4.60 (m, 2H); 6.07 (d, J=13.7 Hz, 1H); 6.58 (d, J=14.4 Hz, 1H); 7.11-7.54 (m, 8H); 8.21 (d, J=13.4 Hz, 1H); 8.42 (d, J=14.2 Hz, 1H).

### Synthesis of the Compound 5 (FIG. 3a)

The coumarin group is reacted with 2-benzyloxy-1,3-dichloropropane, and the adduct is deprotected to give the alcohol bis-coumarin (called multi-B amplifier head) which subsequently reacts with the chloro-cyanine (40). The DOTAGA-ethylenediamine group reacts with the acid function of the intermediate (42) to give the compound (43). The radiolabeling step makes it possible to obtain the compound 5.

### Synthesis of the Compound 6 (FIG. 3b)

Hydroxycoumarin (21 mg, 0.13 mmol) and sodium hydride (6.3 mg, 0.26 mmol) are dissolved in 2 ml of DMF. After 10 min, the compound (40) (50 mg, 0.07 mmol) is added and the course of the reaction is monitored by LCMS. As soon as the reaction is no longer progressing, 20 ml of diethyl ether are added. The solid is filtered off, then washed with diethyl ether and acetone (the purity is monitored by HPLC), to give 11 mg of pure product (41) in the form of a green solid.

One of the six amine functions of the 1,2,3,4,5,6-benzenehexamethanamine is protected while the other five are involved in the reaction with DOTAGA(tBu)₄. The adduct formed (also called multi-A amplifier head) is involved in the reaction with the cyanine. The system obtained is saponified and then radiolabeled with yttrium to give the compound 6.

### 5/ Synthesis of the Compounds 21 and 11 of the Invention (see FIG. 4)

The compounds 21 and 11 are obtained from a symmetrical cyanine (44) bearing two azide groups, said cyanine resulting in the intermediate (45), which itself results in the compound (46) which is a precursor of the compound 21 (FIG. 4a) of the invention or in the compound (48) which is a precursor of the compound 11 (FIG. 4b) of the invention.

### Synthesis of the Precursor (45)

Pyranine (74 mg, 0.14 mmol) and triethylamine (43 mg, 0.42 mmol) are dissolved in 2 ml of dry DMSO. The mixture is stirred at 50° C. for 4 h and then a solution of cyanine (44) (50 mg, 0.07 mmol) in 1 ml of DMSO is added. After 4 h at 45° C., 20 ml of DCM are added to the reaction mixture. After filtration which removes the excess pyranine, and then concentration under reduced pressure, the reaction crude is diluted in water (20 ml) and extracted with diethyl ether (2×30 ml) to give, after lyophilization, 41 mg (54%) of pure product (45) in the form of a green solid.

¹H NMR (500 MHz, methanol-d₄, 300 K); δ (ppm)=0.53 (s, 6H); 1.41 (s, 6H); 1.99 (p, J=6.7 Hz, 4H); 2.10-2.25 (m, 2H); 2.82 (m, 2H); 2.95 (m, 2H); 3.46 (td, J=5.8; 4.0 Hz, 4H); 4.11-4.20 (m, 4H); 6.27 (d, J=14.1 Hz, 2H); 7.06 (t, J=7.5 Hz, 2H); 7.12-7.17 (m, 2H); 7.18 (d, J=8.0 Hz, 2H); 7.24-7.32 (m, 2H); 8.09 (d, J=14.0 Hz, 2H); 8.28 (s, 1H); 9.01 (d, J=9.6 Hz, 1H); 9.23 (s, 2H); 9.50-9.56 (m, 2H).

ESI-HRMS: m/z=1041.2740 [M-2Na⁺ H]⁻ (calculated for C₅₂H₄₉N₈O₁₀S₃⁻: 1041.2739). UV-Vis (water): 241.9; 287.4; 360.0; 394.8; 770.4 nm.

### Synthesis of the Compound 21 (FIG. 4a)

### Synthesis of the Compound (46)

The compound (45) reacts with DOTA-GA-ethylenediamine-BCN according to the following conditions. 6.3 mg of DOTA-GA-ethylenediamine-BCN (0.0129 mmol) are dissolved in 1 ml of phosphate buffer at pH=7.4. Next, 0.48 ml of a solution of cyanine (45) (1.97 mg; 0.00181 mmol) in water is added. The mixture is subsequently stirred at ambient temperature for 3 h and then lyophilized and the crude product obtained is purified by HPLC to give, after lyophilization, 3.55 mg (78%) of target compound (46). UV-Vis (PBS buffer): 242.0; 288.1; 360.5; 395.2; 770.8 nm. HPLC analysis with system A: 3.9 min, 77% MeCN 0.1% FA. HPLC with system B: 25.0 min, 45% MeCN 0.1% FA.

### Preparation of the Compound 21 of the Invention

A conjugation step (verification of pH, temperature, antibody concentration) makes it possible to obtain the “compound 46-antibody” system, that is to say the compound (47). The bioconjugate is radiolabeled with Y90 to give the compound 21 of the invention.

### Synthesis of the Compound 11 (FIG. 4b)

### Synthesis of the Compound (48)

The cyanine (45) is dissolved in 2 ml of dry DMSO and then the DOTA-GA-ethylenediamine-BCN is added. The mixture is stirred at 50° C. for 16 h. The compound (48) is obtained.

¹H NMR (500 MHz, methanol-d₄, 300 K): δ (ppm)=0.53 (s, 6H); 1.41 (s, 6H); 1.99 (p, J=6.7 Hz, 4H); 2.10-2.25 (m, 2H); 2.82 (m, 2H); 2.95 (m, 2H); 3.46 (td, J=5.8; 4.0 Hz, 4H); 4.11-4.20 (m, 4H); 6.27 (d, J=14.1 Hz, 2H); 7.06 (t, J=7.5 Hz, 2H); 7.12-7.17 (m, 2H); 7.18 (d, J=8.0 Hz, 2H); 7.24-7.32 (m, 2H); 8.09 (d, J=14.0 Hz, 2H); 8.28 (s, 1H); 9.01 (d, J=9.6 Hz, 1H); 9.23 (s, 2H); 9.50-9.56 (m, 2H). ESI-HRMS: m/z=1041.2740 [M-2Na⁺ H]⁻ (calculated for C₅₂H₄₉N₈O₁₀S₃⁻: 1041.2739). UV-Vis (water): 241.9; 287.4; 360.0; 394.8; 770.4 nm.

### Synthesis of the Compound 11

The bismacrocyclic compound (48) in a buffer is incubated for several hours in the presence of a defined amount (MBq) of yttrium-90 trichloride ⁹⁰YCl₃ in order to achieve the desired specific activity. The radiochemical purity is verified by RI-TLC.

The compound 11 of the invention is obtained.

### 6/ Synthesis of the Compounds 4 and 19 of the Invention (See FIG. 5)

### Synthesis of the Compound (49)

The coumarin (646 mg, 4 mmol) and 4,5-dichlorophthalo-nitrile (788 mg, 4 mmol) are dissolved in 10 ml of DMF (dimethylformamide) in the presence of K₂CO₃ (2.21 g, 16 mmol). The resulting mixture is stirred at 45° C. for 16 h and is then recovered by filtration. The filtrate is concentrated under reduced pressure and purified by column chromatography using a DCM/MeOH 9(5/5 vol.) solvent mixture as eluent, to give 920 mg (70%) of the desired compound (49) in the form of a powder.

¹H NMR (500 MHz, CDCl₃, 300 K): δ (ppm)=6.45 (d, J=9.6 Hz, 1H); 6.97 (dd, J=8.5; 2.4 Hz, 1H); 7.02 (d, J=2.4 Hz, 1H); 7.26 (s, 1H); 7.59 (d, J=8.4 Hz, 1H); 7.73 (d, J=9.6 Hz, 1H); 7.94 (s, 1H).

¹³C NMR (125 MHz, CDCl₃, 300 K): δ (ppm)=108.17; 111.80; 114.06; 114.16; 115.77; 115.97; 116.68; 117.02; 122.79; 130.16; 131.19; 136.13; 142.55; 155.74; 156.37; 156.60; 159.82.

MALDI-TOF (calculated for C₁₇H₇ClN₂O₃: 322.0145, found 323).

### Synthesis of the Phthalonitrile-Coumarin-Pyridine System (50)

The phthalonitrile-coumarin conjugate (40) (400 mg, 1.24 mmol), 4-hydroxypyridine (176 mg, 1.85 mmol) and K₂CO₃ (512 mg, 71 mmol) are dissolved in 20 ml of DMF. The resulting mixture is stirred at 45° C. for 16 h. Next, K₂CO₃ is separated by filtration on a frit and then the filtrate is concentrated under reduced pressure and purified by chromatography using the (DCM/MeOH 90/10) solvent mixture as eluent, to give 200 mg (%) of the desired product (50).

### Synthesis of the Diiminoisoindoline-Coumarin-Pyridine System (51)

A solution of dicyanobenzene (50) in methanol is brought to reflux with ammonia bubbling for several hours. After distillation of the solvent, the compound obtained (51) is immediately used in the next synthesis step.

### Steps for Converting (51) into the Compounds 4 and then 19 of the Invention

The diiminoisoindoline (51) is reacted with a silicon salt. The reaction mixture is brought to reflux and then the solvent is distilled under reduced pressure. The residue obtained is washed with a series of solvents, then dried and immediately used in the following step. The intermediate (52) reacts with 3-azidoethanol in the presence of a base and brought to reflux. After purification, a suspension of phthalocyanine (53) in methyl iodide is brought to reflux. At the outcome, the excess methyl iodide is distilled and the phthalocyanine (54) is purified by semipreparative HPLC. The intermediate (54) and DOTAGA-ethylenediamine-BCN are reacted; after distillation of the solvents, the target conjugate (55) is separated from the compound (54) and the by-products by HPLC.

### EXAMPLE 2

Use of the Compounds of the Invention for Cherenkov-PDT

An in-tube in vitro study and in vitro study on cells makes it possible to show the properties of transfer by CRET/TBET or CRET/FRET, which is intramolecular, of the compounds of the invention.

The reactive oxygen species (ROSs) are measured by UV/Visible spectrometry by following the disappearance of the DPBF (diphenylbenzofuran) absorbance band following the reaction with the ROSs generated by the Cherenkov photodynamic process.

In Vitro Study on Cells

The cells are plated onto 96-well microplates, and incubated with the solution of a compound of the invention in the total absence of parasitic exogenous light source capable of exciting the photosensitizing compound.

A control plate is prepared in the presence of the non-radiolabeled compound in order to prove that the toxicity measured does not come:
from a resulting parasitic cytotoxic effect,
from the intrinsic cytotoxicity of the compound,
from a photocytotoxicity resulting from excitation by an exogenous light source.

Moreover, a control study using the non-radiolabeled parent compound—and in the absence of exogenous light source—makes it possible to confirm the origin of the cytotoxicity.

In Vivo Cherenkov-PDT Protocol

It begins with the intravenous injection of a compound of the invention into xenografted mice carrying a deep cancer model of cancer cells.

A control batch of mice injected with a radiolabeled bioconjugate, of AD structure, that is to say not comprising BC, is prepared. The tumor volume is monitored by carrying out PET imaging of the tumor. This imaging is carried out in the following way: the mice are anesthetized then injected with ¹⁸F-fluorodeoxyglucose (¹⁸F-FDG) and are subsequently imaged on a μPET imager. Throughout the experiment, all precautions are taken so that no parasitic light, that is to say light other than the Cherenkov radiation, can reach the tumor labeled with the compound of the invention.

The following verifications are carried out:
the tumor is deep (>1 cm deep),
the mice have hair, and optionally a screen can be affixed on the study zone.

The control mouse batch studied makes it possible to show the change in the tumor volume in the case of prolonged exposure to an exogenous, and potentially parasitic, light source. At a sufficient depth, the change in tumor volume for control mice versus treated mice makes it possible to demonstrate the efficacy of the compounds of the invention.

### EXAMPLE 3

Use of the Compounds of the Invention for Near-IR CLI

CLI Protocol

A study on an optical imager makes it possible to measure the properties of transfer by CRET/TBET or CRET/FRET, which is intramolecular, of the compound 1 of the invention.

Moreover, an in vitro study showed that the non-radiolabeled parent compound does not exhibit any cytotoxicity.

The CLI protocol begins with the intravenous injection of the compound 1 of the invention into xenografted mice carrying a tumor.

A control batch of mice injected with a radiolabeled bioconjugate of AD type, that is to say not comprising BC, is also prepared.

When the AD bioconjugate has reached the tumor, after several hours (the number of hours will depend on the nature of the biomolecule), the mice are anesthetized and are then placed in the optical imager.

The mice are imaged in Cherenkov mode and in bioluminescence mode, first by examining the radiance over the entire spectral window of the optical imager (500-850 nm) and then by using filters in order to examine the radiance on the near-infrared zone exclusively.

At the end of the experiments, the mice are sacrificed.

The radiance measurement is the step which makes it possible to demonstrate the transfer to the near-infrared and the efficacy of the compounds of the invention. This involves a direct comparison of the radiance between the batch of control mice injected with AD (the radiolabeled biomolecule, that is to say the Cherenkov radiation alone, not amplified by the BC antenna), and the batch of mice injected with the compound 1 of the invention.

The comparison of the result obtained with the compound 1 of the invention and the result obtained by the authors Bernhard et al.⁽⁷⁾ makes it possible to demonstrate the relevance of a single-molecule probe rather than a multimolecular probe, and the advantage of the compounds of the invention.

### LITERATURE REFERENCES

(1) Grootendorst, M. R., Cariati, M., Kothari, A., Tuch, D. S., Purushotam, A. Cerenkov luminescence imaging (CLI) for image-guided cancer surgery, Clin. Transl. Imaging (2016). 4, 353-366.
(2) Demian van Straten, Vida Mashayekhi, Henriette S. de Bruijn, Sabrina Oliveira and Dominic J. Robinson, Oncologic Photodynamic Therapy: Basic Principles, Current Clinical Status and Future Directions, Cancers, 2017, 9. 19, 1-54, doi:10.3390/cancers 9020019.
(3) Robertson, R. et al. Optical imaging of Cerenkov light generation from positron-emitting radiotracers. Physics Medicine Biology 54, N355-N365 (2009).
(4) Dothager, R. S., Goiffon, R. J., Jackson, E., Harpstrite, S. & Piwnica-Worms, D. Cerenkov Radiation Energy Transfer (CRET) Imaging: A Novel Method for Optical Imaging of PET Isotopes in Biological Systems. PLoSone 5, c13300 (2010).
(5) Boschi, F. & Spinelli. A. E. Quantum clots excitation using pure beta minus radioisotopes emitting Cerenkov radiation. RSC Advance 2, 11049-1052 (2012).
(6) Bernhard et al., Chemical Communications, 2014, 50, pp. 6711-6713.
(7) Bernhard et al., Scientific Reports, 2017, 7, 45063.
(8) Bizet et al., Bioorganic & medicinal Chemistry 26 (2018), pp. 413-420.
(9) Anyanee KamKaew et al., Applied Materials & Interfaces, 2016, 8 (40), pp 26630-26637.
(10) C. Göl, M. Malkoç, S. Yeşilot, M. Durmuş, Dyes Pigm, 111 (2014), pp. 81-90.
(11) S. Osati, H. Ali, J. E. van Lier, Tetrahedron Lett, 56 (2015), pp. 2049-2053,
(12) H. Yanik, M. Göksel, S. Yesilot, M. Durmus, Tetrahedron Lett, 57 (2016), pp. 2922-2926.
(13) A. Loudet, C. Thivierge, K. Burgess, Dojin News (2011), p. 137, http://www.dojindo.co.jp/letterj/137, review/01.html.
(14) Wen-Hai Zhan, Jian-Li Hua, Ying-Hua Jin, Xin Teng, and He Tian, Res. Chem. Intermed., Vol. 34, No. 2-3, pp. 229-239 (2008).

1-10. (canceled)

11. A compound having the following general structure (I): 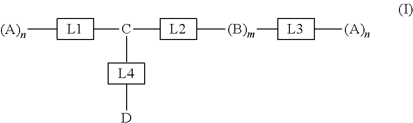
wherein:
A is a radioactive entity which is a beta-energy emitter which produces Cherenkov radiation, said radioactive entity preferably being a radiochelate, namely a radiometal surrounded by a chelate or a radioelement which is non-metallic;
B is a fluorophore which absorbs electromagnetic radiation of a wavelength λ ranging from 300 nm to 500 nm;
C is a fluorophore which emits electromagnetic radiation of a wavelength λ ranging from 650 nm to 950 nm,
and/or
C is a photosensitizer which produces reactive oxygen species ROSs;
D may be present or absent, and represents, when it is present, a vector entity, said vector entity preferably being a biomolecule or a nanoparticle vector,
L1, L2, L3 are each, independently of one another, an at least divalent linking group or a covalent bond, with the condition that 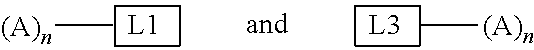
are not simultaneously present, which means that, if 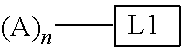
is present, then 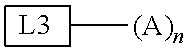
is absent and vice versa,
L4 is present if D is present and represents, when it is present, an at least divalent linking group or a covalent bond;
n is an integer equal to 1, 2, 3, 4 or 5;
m is an integer equal to 1, 2, 3, 4, 5, 6, 7 or 8;
and wherein:
A activates B by CRET-type energy transfer,
B transfers the energy received from A to C, by intramolecular FRET-type energy transfer or by TBET-type energy transfer.

12. The compound as claimed in claim 11, having the following structure (I-1): 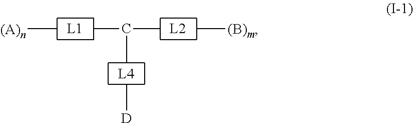
wherein A, B, C, D, L1, L2, L4, n and m are as defined above.

13. The compound as claimed in claim 11, having the following structure (I-2): 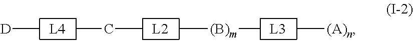
wherein A is a non-metallic radioelement, and B, C, D, L2, L3, L4, n and m are as defined above.

14. The compound as claimed in claim 11, wherein A is:
a radiochelate, the radiometal of which is chosen from the group comprising ⁹⁰Y ¹⁷⁷Lu, ⁶⁹Ga, ⁸⁹Zr, ⁶⁴Cu, ⁸⁹Sr, ²¹²Bi, ²¹³Bi, ⁴⁴Sc, ²²⁵Ac and ⁴⁴Sc and the chelating agent of which is chosen from the group comprising DOTAGA, DOTA, NOTA, NODAGA and DFO,
a non-metallic radioelement chosen from the group comprising ¹⁸F, ¹³¹L ¹²⁴I and ³²P.

15. The compound as claimed in claim 11, wherein B is chosen from the group comprising a nucleus of the type coumarin; substituted coumarin, in particular substituted with one or more hydroxyls and/or with a pyridinium, itself optionally substituted; pyranine; pyrene; BODIPY; substituted BODIPY, in particular phenyl-BODIPY, hydroxyphenyl-BODIPY, aza-BODIPY; fluorescein; rhodamine, in particular rhodamine 6G, rhodamine 101, rhodamine B, rhodamine 123; eosin, in particular eosin B, eosin Y; tryptophan, and mixtures thereof.

16. The compound as claimed in claim 11, wherein C is chosen from the group comprising a nucleus of the cyanine type, in particular cyanine-7, cyanine-5, cyanine-3; phthalocyanine, in particular silicon, zinc, magnesium, phosphorus, aluminum, indium phthalocyanine, naphthalocyanine, in particular zinc, magnesium, phosphorus, aluminum, silicon, indium naphthalocyanine; chlorin, in particular zinc, magnesium, phosphorus, aluminum, silicon, indium chlorin; bacteriochlorin, in particular zinc, magnesium, phosphorus, aluminum, silicon, indium bacteriochlorin.

17. The compound as claimed in claim 15, wherein B and/or C comprise(s) at least one solubilizing group chosen from the group comprising a sulfonate (SO₃⁻); a carboxylate (COO⁻); an ammonium (NR₄⁺) with R═H, alkyl or aryl; a phosphonate (PO₃²⁻); a pyridinium, which is preferably substituted; an imidazolium, and mixtures thereof.

18. The compound as claimed in claim 11, wherein D is a biomolecule, in particular a peptide; a protein; a protein of antibody type; a protein of antibody fragment type, such as Fab, Fab′2, Fab′, ScFv, nanobody, affibody, diabody; an aptamer.

19. A method for near-infrared Cherenkov luminescence imaging, comprising administering to a subject a compound as claimed in claim 11.

20. A method of treating deep biological tissues by Cherenkov photodynamic therapy, comprising administering to a subject in need thereof a compound as claimed in claim 11.

21. The compound as claimed in claim 16, wherein B and/or C comprise(s) at least one solubilizing group chosen from the group comprising a sulfonate (SO₃⁻); a carboxylate (COO⁻); an ammonium (NR₄⁺) with R═H, alkyl or aryl; a phosphonate (PO₃²⁻); a pyridinium, which is preferably substituted; an imidazolium, and mixtures thereof.
